(19) United States
(12) Patent Application Publication
(10) Pub. No.: US 2024/0368112 A1
(43) Pub. Date: Nov. 7, 2024

(54) BENZYLTHIOPHENE DERIVATIVES

(71) Applicants: Rosalind Franklin University of Medicine & Science, North Chicago, IL (US); University of Tennessee Health Science Center, Memphis, TN (US); Cedars-Sinai Medical Center, Los Angeles, CA (US)

(72) Inventors: John K. Buolamwini, North Chicago, IL (US); Mithun Raje, North Chicago, IL (US); Hemantkumar Deokar, North Chicago, IL (US); Lawrence Pfeffer, Memphis, TN (US); James Turkson, Los Angeles, CA (US)

(21) Appl. No.: 18/689,598

(22) PCT Filed: Sep. 16, 2022
(86) PCT No.: PCT/US2022/043775

§ 371 Date: Mar. 6, 2024

Related U.S. Application Data
(60) Provisional application No. 63/249,194, filed on Sep. 28, 2021.

(51) Int. Cl. C07D 333/38; A61K 31/381; A61P 35/00

(52) U.S. Cl. CPC C07D 333/38; A61K 31/381; A61P 35/00

(57) ABSTRACT

Disclosed are benzylthiophene derivatives, pharmaceutical compositions comprising them, and methods of using them.

### CROSS-REFERENCE TO RELATED APPLICATIONS

This application is a section 371 U.S. national phase of PCT/US2022/043775, filed Sep. 16, 2022, which claims the benefit of priority of U.S. Provisional Application No. 63/249,194, filed Sep. 28, 2021, both which are incorporated herein by reference in their entirety.

### STATEMENT REGARDING FEDERALLY SPONSORED RESEARCH

The claimed invention was made with U.S. Government support under grant number 1R01CA208851 awarded by the National Science Foundation (NSF) and National Cancer Institute (NCI). The government has certain rights in this invention.

### BACKGROUND OF THE DISCLOSURE

### Field of the Disclosure

This disclosure relates generally to benzylthiophene-derived molecules, pharmaceutical compositions comprising them, and methods of using them.

### Technical Background

Signal transducer and activator of transcription 3 (STAT3) is a potential drug target for cancer and inflammation. STAT3 is a protein that functions as a transcription factor for genes related to cell growth, survival, proliferation, differentiation, and apoptosis. STAT3 activation happens when a phosphorus group is added to a tyrosine residue at the SH2 domain of STAT3 by various positive effectors such as janus kinases and tyrosine kinases. As the result, dimers are formed that translocate to the nucleus where they bind to promoter regions of the genes and activate gene expression. STAT3 suppresses apoptosis, upregulates the expression of cell proliferation genes, stimulates tumor angiogenesis, and opposes anti-tumor immune responses. There have been attempts to inhibit the dimerization process but unfortunately they were not successful. The strategies to block STAT3 dimerization have dominated STAT3 inhibitor discovery. Accordingly, there is a need for the design and development of new STAT3 inhibitors.

### SUMMARY OF THE DISCLOSURE

One aspect of the disclosure provides compounds having the structural formula:

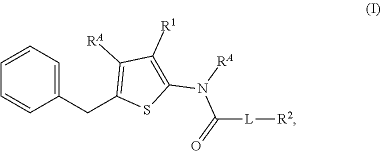

or a pharmaceutically acceptable salt thereof,
wherein:
each R^{A} is independently H or methyl;
R¹ is —CN or —C(O)—N(R^{A})₂;
R² is aryl or heteroaryl, wherein the aryl or heteroaryl is monocyclic or bicyclic and comprised of 5-10 ring members, and optionally substituted with 1-4 R^{B}, wherein R^{B} is C₁-C₂ alkyl, fluoro, chloro, or —O—(C₁-C₂ alkyl), wherein each alkyl is optionally fluorinated.
L is absent or —C(R^{A})₂—O—.

In another aspect, the disclosure provides pharmaceutical compositions comprising a compound (e.g., a compound of formula (I)) as described herein.

In another aspect, the disclosure provides methods for treating various diseases, such as cancer, to a subject in need thereof. The methods include administering to the subject a therapeutically effective amount of a compound as described herein (e.g., a compound of formula (I)).

Other aspects and embodiments of the disclosure are evident in view of the detailed description provided herein.

### BRIEF DESCRIPTION OF THE DRAWINGS

FIG. 1: Effects of Compound 1 and analogs on STAT3 phosphorylation in MT330 GBM cells. MT330 cells were treated with Compound 1 (first panel), Compound 5 (second panel) or Compound 3 (third panel) for 24 hr at indicated concentrations and then treated with IFNα (1000 U/ml) for 30 min and the expression of STAT3, pSTAT3, and pSTAT1 proteins was determined by immunoblotting.

FIG. 2: Antiproliferative Activity of Compound 1 against (A) MT300 (B) LN 229 and GBM cell lines. Cells were treated with Compound 1 at varying concentrations for 72 h and cell proliferation was measured by the CellTiter Glo assay.

FIG. 3: A and B: The sensitivity of STAT3-KO MT330 GBM cells to Compound 1. (A) STAT3-KO MT330 cells were restored with either wild-type or F705Y-STAT3 and then treated with IFNα (IFN) (1000 U/ml for 30 min). Lysates were prepared and immunoblotted as indicated. Proliferation of (B) EV (empty vector) treated or (C) STAT3-KO MT330 cells in the presence or absence of the indicated concentration of Compound 1 was measured with the Incucyte live cell analysis system.

FIG. 4: Human astrocyte cells were treated with Compound 1, DP3 or DP5 for 24 hr at the indicated concentrations and then treated with IFNα (1000 U/ml for 30 min) and the expression of STAT3, pSTAT3, and pSTAT1 was determined by immunoblotting.

FIG. 5: The binding isotherm from the integrated thermogram fit using the one-site model was used to extract the thermodynamic parameters of Compound 1 binding to STAT3.

FIG. 6: The anti-glioma effects of the STAT3 inhibitor Compound 1. Mice were intracranially injected with 1×10⁵ luciferase-tagged GBM6 GSCs and intraperitoneally injected with Compound 1 (20 mg/kg) every other day. BLI pictures were taken at Day 21.

FIG. 7 depicts the STAT3 signaling pathway.

FIG. 8 depicts MCF7 cells before (upper) and after (lower) treatment with 20 μM of Compound 7 according to an example embodiment.

FIG. 9 depicts the calculated docking of Compound 4 to the STAT3 active site according to an example embodiment.

### DETAILED DESCRIPTION

The disclosure provides compounds, pharmaceutical compositions, methods and uses for treating a variety of diseases associated with STAT3 inhibition, such as cancer.

Signal transducers and activators of transcription (STATs) regulate gene expression in normal cellular responses to cytokines and growth factors. STAT3 is the most studied one of the 7 family members identified due to its major mediatory effects on carcinogenesis and inflammation. The aberrant activation of STAT3 has been detected in a wide variety of human cancer cell lines and tissues.

Constitutively active STAT3 suppresses apoptosis, upregulates the expression of cell proliferation genes, stimulates tumor angiogenesis, and opposes anti-tumor immune responses. Hence, STAT3 is considered as a promising therapeutic target for cancer therapy.

STAT 3 activation begins with the phosphorylation of a critical tyrosine residue (Tyr705) on its Src homology 2 (SH2) domain by activated growth factor receptors, Janus kinases (JAKs) or Src tyrosine kinase. Upon activation, STAT3 forms dimers through a reciprocal phosphotyrosine (pTyr705):SH2 domain interaction and translocates to the nucleus where the dimers bind to the promoters of target genes and activate specific gene expression. For many years, the SH2 domain-pTyr dimerization step was regarded as an attractive target for interfering with STAT3 function and this strategy has been exploited in many STAT3 drug discovery approaches. Most nonpeptide STAT3 inhibitors known to date are small molecule compounds and peptidomimetics, and are mainly STAT3 dimerization disrupting agents. Unfortunately, none of these STAT3 dimerization disrupting agents has reached the clinic as cancer therapeutics. In contrast to STAT3 dimerization disrupting agents, not much progress has been made in approaches aimed at direct inhibition of STAT3 DNA binding. Recent studies have indicated that STAT3 nuclear import can take place constitutively and independently of Tyr705 phosphorylation through the nuclear import carrier importin-α3. In addition, the binding of unphosphorylated STAT3 directly to DNA has also been observed by an electrophoretic mobility shift assay. These studies suggest that the strategy of blocking STAT3 dimerization by targeting the SH2 domain might be unable to effectively inhibit STAT3 activity. Although a number of molecules have been screened or developed as putative STAT3 SH2 domain binding inhibitors, binding sites for these small molecules at the SH2 domain have not been identified by any crystal structure. The inability of any of the many small molecule STAT3 SH2 domain targeting drugs to progress to the clinic gives cause to pursue other STAT3 domain binding inhibitors.

Accordingly, one aspect of the disclosure provides compounds having the structural formula:

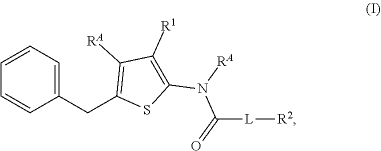

or a pharmaceutically acceptable salt thereof,
wherein:
each R^{A} is independently H or methyl;
R¹ is —CN or —C(O)—N(R^{A})₂;
R² is aryl or heteroaryl, wherein the aryl or heteroaryl is monocyclic or bicyclic and comprised of 5-10 ring members, and optionally substituted with 1-4 R^{B}, wherein R^{B} is C₁-C₂ alkyl, fluoro, chloro, or —O—(C₁-C₂ alkyl), wherein each alkyl is optionally fluorinated.
L is absent or —C(R^{A})₂—O—.

In certain embodiments as otherwise described herein, R¹ is —CN or —C(O)—NH₂. For example, in particular embodiments, R¹ is —CN.

In certain embodiments as otherwise described herein, R² is phenyl or benzofuranyl. For example, in particular embodiments, R² is phenyl.

In certain embodiments as otherwise described herein, R² is unsubstituted. In other embodiments, R² is substituted with 1-2 R^{B}, wherein R^{B} is CH₃, CF₃, chloro, —O—CH₃, or —O—CF₃.

In certain embodiments as otherwise described herein, L is —C(R^{A})₂—O—. In other embodiments, L is —C(R^{A})₂—O—. For example, in particular embodiments, L is —C(CH₃)₂—O— or —CH₂—O—.

In another aspect, the disclosure provides pharmaceutical compositions comprising a compound (e.g., a compound of formula (I)) as described herein.

In another aspect, the disclosure provides methods for treating various diseases, such as cancer, to a subject in need thereof. The methods include administering to the subject a therapeutically effective amount of a compound as described herein (e.g., a compound of formula (I)).

In certain embodiments of the compounds as otherwise described herein, the compound is not 5-benzyl-2-(2-(2-methoxyphenoxy)acetamido)-4-methylthiophene-3-carboxamide, N-[3-carbamoyl-5-benzyl-thien-2-yl]4-hydroxyphenoxyacetamide, or 5-benzyl-4-methyl-2-(2-phenoxyacetamido)thiophene-3-carboxamide.

In certain additional embodiments, including any of the embodiments described with reference to formula (I) above, each optionally substituted alkyl, alkenyl, and alkynyl recited in any one of preceding embodiments is unsubstituted.

In certain additional embodiments, including any of the embodiments described with reference to formula (I) above, each aryl is phenyl.

In certain additional embodiments, including any of the embodiments described with reference to formula (I), each heteroaryl is a 5-10 membered monocyclic or bicyclic heteroaryl having 1-3 heteroatoms selected from O, S and N. For example, in certain particular embodiments, each heteroaryl is a bicyclic heteroaryl substituted with O or N. For example, in certain particular embodiments, each heteroaryl is a bicyclic heteroaryl is substituted with 0-3 R^{B}, e.g., is unsubstituted, substituted with one R^{B} or substituted with two R^{B}. In certain particular embodiments each heteroaryl is benzofuranyl.

In certain additional embodiments, including any of the embodiments described with reference to formula (I) above and any embodiment described in the paragraphs immediately above, each R^{B} is independently methyl, fluoro, chloro, methoxy, or —CF₃. For example, in certain additional embodiments, each R^{C} is independently methyl, or CF₃.

In certain embodiments of the compounds as otherwise described herein, the compound is in the form of a pharmaceutically acceptable salt of a compound as described herein. The person of ordinary skill in the art will appreciate that a variety of pharmaceutically-acceptable salts may be provided, as described in additional detail below. In certain embodiments of the compounds as otherwise described herein, a compound is in the form of a solvate (e.g., a hydrate) of a compound or salt as described herein. The person of ordinary skill in the art will appreciate that a variety of solvates and/or hydrates may be formed. The person of ordinary skill in the art will appreciate that the phrase “optionally in the form of a pharmaceutically acceptable salt thereof, and/or a solvate or hydrate thereof” includes compounds in the form of solvates and hydrates of base compounds or pharmaceutically acceptable salts as described above. But in certain embodiments as described above, the compound is not in the form of a solvate or hydrate.

### Therapeutic Applications

The disclosure also provides methods of inhibiting STAT3 activation in a subject in need thereof. For example, in certain embodiments, the subject may have cancer. Representative cancers include breast cancer, glioblastoma multiforme brain cancer, colorectal cancer, lung cancer, pancreatic cancer, bladder cancer, and metastatic prostate cancer. These methods include administering to a subject in need of such treatment an effective amount of one or more compounds of the disclosure as described herein (e.g., compounds of formula (I)) or a pharmaceutical composition of the disclosure as described herein.

In certain embodiments, the present disclosure provides for a method of treating a condition in a subject in need thereof, wherein the method includes providing to the subject a compound as otherwise described herein.

### Pharmaceutical Compositions and Dosage Forms

A compound as described herein can usefully be provided in the form of a pharmaceutical composition. Such compositions include the compound according to any one of the preceding aspects or embodiments described herein, together with a pharmaceutically acceptable excipient, diluent, or carrier.

The compounds may be formulated in the pharmaceutical composition per se, or in the form of a hydrate, solvate, or pharmaceutically acceptable salt, as previously described. Typically, such salts are more soluble in aqueous solutions than the corresponding free acids and bases, but salts having lower solubility than the corresponding free acids and bases may also be formed.

The pharmaceutical composition can be, for example, in the form of a tablet, a capsule, or a parenteral formulation, but the person of ordinary skill in the art will appreciate that the compound can be provided in a wide variety of pharmaceutical compositions.

The compounds of the disclosure can be administered, for example, orally, topically, parenterally, by inhalation or spray or rectally in dosage unit formulations containing one or more pharmaceutically acceptable carriers, diluents or excipients. The term parenteral as used herein includes percutaneous, subcutaneous, intravascular (e.g., intravenous), intramuscular, or intrathecal injection or infusion techniques and the like. A medicament including a compound of the disclosure can be provided in any appropriate of the formulations and dosage forms as described herein.

Pharmaceutical compositions can be made using the presently disclosed compounds. For example, in one embodiment, a pharmaceutical composition includes a pharmaceutically acceptable carrier, diluent or excipient, and compound as described above with reference to any one of structural formulae.

In the pharmaceutical compositions disclosed herein, one or more compounds of the disclosure may be present in association with one or more pharmaceutically acceptable carriers, diluents or excipients, and, if desired, other active ingredients. The pharmaceutical compositions containing compounds of the disclosure may be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any suitable method for the manufacture of pharmaceutical compositions and such compositions may contain one or more agents selected from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by suitable techniques to delay disintegration and absorption in the gastrointestinal tract and thereby provide a sustained action over a longer period. For example, a time delay material such as glyceryl monostearate or glyceryl distearate can be employed.

Formulations for oral use can also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil. Formulations for oral use can also be presented as lozenges.

Aqueous suspensions contain the active materials in admixture with excipients suitable for the manufacture of aqueous suspensions. Such excipients can be suspending agents, for example sodium carboxymethylcellulose, methylcellulose, hydropropylmethylcellulose, sodium alginate, polyvinylpyrrolidone, gum tragacanth and gum acacia; dispersing or wetting agents such as a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

In some embodiments, the pharmaceutically acceptable carrier, diluent, or excipient is not water. In other embodiments, the water comprises less than 50% of the composition. In some embodiments, compositions comprising less than 50% water have at least 1%, 2%, 3%, 4% or 5% water. In other embodiments, the water content is present in the composition in a trace amount.

In some embodiments, the pharmaceutically acceptable carrier, diluent, or excipient is not alcohol. In other embodiments, the alcohol comprises less than 50% of the composition. In some embodiments, compositions comprising less than 50% alcohol have at least 1%, 2%, 3%, 4% or 5% alcohol. In other embodiments, the alcohol content is present in the composition in a trace amount.

Syrups and elixirs can be formulated with sweetening agents, for example glycerol, propylene glycol, sorbitol, glucose or sucrose. Such formulations can also contain a demulcent, a preservative, flavoring, and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils can be employed as a solvent or suspending medium. For this purpose any bland fixed oil can be employed including synthetic mono- or diglycerides. In addition, fatty acids such as oleic acid find use in the preparation of injectables.

Compounds of the disclosure can also be administered in the form of suppositories, e.g., for rectal administration of the drug. These compositions can be prepared by mixing the compound with a suitable non-irritating excipient that is solid at ordinary temperatures but liquid at the rectal temperature and will therefore melt in the rectum to release the drug. Such materials include cocoa butter and polyethylene glycols.

Compounds of the disclosure can also be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

The compositions can be formulated in a unit dosage form of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound described herein. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, 0.1 to about 500 mg of the active ingredient of a compound described herein.

The tablets or pills can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like. Suitable representative ranges of therapeutically effective dosages may be in the range of 0.1 to 2,000 mg/day based on an adult subject with a 70 kg body weight. The compound as otherwise described herein may be administered once daily or in two, three, four, or six divided doses. The dosage may vary depending on health conditions, age, body weight, sex, administration route, and severity of illness.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds can vary according to, for example, the particular use for which the treatment is made, the manner of administration of the compound, the health and condition of the patient, and the judgment of the prescribing physician. The proportion or concentration of a compound described herein in a pharmaceutical composition can vary depending upon a number of factors including dosage, chemical characteristics (e.g., hydrophobicity), and the route of administration. For example, the compounds described herein can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compounds described herein can also be formulated in combination with or administered sequentially with one or more additional active ingredients which can include any pharmaceutical agent such as anti-viral agents, vaccines, antibodies, immune enhancers, immune suppressants, anti-inflammatory agents and the like.

The person of ordinary skill in the art will formulate a compound as described into pharmaceutical formulations herein. For example, based on the physicochemical properties of the compound, one of ordinary skill in the art will recognize a pharmaceutically effective amount of the compound, and the desired route of administration.

### Definitions

Terms used herein may be preceded and/or followed by a single dash, “—”, or a double dash, “═”, to indicate the bond order of the bond between the named substituent and its parent moiety; a single dash indicates a single bond and a double dash indicates a double bond or a pair of single bonds in the case of a spiro-substituent. In the absence of a single or double dash it is understood that a single bond is formed between the substituent and its parent moiety; further, substituents are intended to be read “left to right” with reference to the chemical structure referred to unless a dash indicates otherwise. For example, arylalkyl, arylalkyl-, and -alkylaryl indicate the same functionality.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety can refer to a monovalent radical (e.g., CH₃—CH₂—), in some circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene). All atoms are understood to have their normal number of valences for bond formation (i.e., 4 for carbon, 3 for N, 2 for O, and 2, 4, or 6 for S, depending on the oxidation state of the S). Nitrogens in the presently disclosed compounds can be hypervalent, e.g., an N-oxide or tetrasubstituted ammonium salt. On occasion a moiety may be defined, for example, as —B-(A)ₐ, wherein a is 0 or 1. In such instances, when a is 0 the moiety is —B and when a is 1 the moiety is —B-A.

As used herein, the term “alkyl” includes a saturated hydrocarbon having a designed number of carbon atoms, such as 1 to 10 carbons (i.e., inclusive of 1 and 10), 1 to 8 carbons, 1 to 6 carbons, 1 to 3 carbons, or 1, 2, 3, 4, 5 or 6. Alkyl group may be straight or branched and depending on context, may be a monovalent radical or a divalent radical (i.e., an alkylene group). For example, the moiety “—(C₁-C₆ alkyl)-O—” signifies connection of an oxygen through an alkylene bridge having from 1 to 6 carbons and C₁-C₃ alkyl represents methyl, ethyl, and propyl moieties. Examples of “alkyl” include, for example, methyl, ethyl, propyl, isopropyl, butyl, iso-, sec- and tert-butyl, pentyl, and hexyl.

The term “alkoxy” represents an alkyl group of indicated number of carbon atoms attached to the parent molecular moiety through an oxygen bridge. Examples of “alkoxy” include, for example, methoxy, ethoxy, propoxy, and isopropoxy.

The term “aryl” represents an aromatic ring system having a single ring (e.g., phenyl) which is optionally fused to other aromatic hydrocarbon rings or non-aromatic hydrocarbon or heterocycle rings. “Aryl” includes ring systems having multiple condensed rings and in which at least one is carbocyclic and aromatic, (e.g., 1,2,3,4-tetrahydronaphthyl, naphthyl). Examples of aryl groups include phenyl, 1-naphthyl, 2-naphthyl, indanyl, indenyl, dihydronaphthyl, fluorenyl, tetralinyl, and 6,7,8,9-tetrahydro-5H-benzo [a] cycloheptenyl. “Aryl” also includes ring systems having a first carbocyclic, aromatic ring fused to a nonaromatic heterocycle, for example, 1H-2,3-dihydrobenzofuranyl and tetrahydroisoquinolinyl. The aryl groups herein are unsubstituted or, when specified as “optionally substituted”, can unless stated otherwise be substituted in one or more substitutable positions with various groups as indicated.

The terms “halogen” or “halo” indicate fluorine, chlorine, bromine, and iodine. In certain embodiments of each and every embodiment as otherwise described herein, the term “halogen” or “halo” refers to fluorine or chlorine. In certain embodiments of each and every embodiment described herein, the term “halogen” or “halo” refers to fluorine.

The term “heteroaryl” refers to an aromatic ring system containing at least one aromatic heteroatom selected from nitrogen, oxygen and sulfur in an aromatic ring. Most commonly, the heteroaryl groups will have 1, 2, 3, or 4 heteroatoms. The heteroaryl may be fused to one or more non-aromatic rings, for example, cycloalkyl or heterocycloalkyl rings, wherein the cycloalkyl and heterocycloalkyl rings are described herein. In one embodiment of the present compounds the heteroaryl group is bonded to the remainder of the structure through an atom in a heteroaryl group aromatic ring. In another embodiment, the heteroaryl group is bonded to the remainder of the structure through a non-aromatic ring atom. Examples of heteroaryl groups include, for example, pyridyl, pyrimidinyl, quinolinyl, benzothienyl, indolyl, indolinyl, pyridazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, benzo[1,4]oxazinyl, triazolyl, tetrazolyl, isothiazolyl, naphthyridinyl, isochromanyl, chromanyl, isoindolinyl, isobenzothienyl, benzoxazolyl, pyridopyridinyl, purinyl, benzodioxolyl, triazinyl, pteridinyl, benzothiazolyl, imidazopyridinyl, imidazothiazolyl, benzisoxazinyl, benzoxazinyl, benzopyranyl, benzothiopyranyl, chromonyl, chromanonyl, pyridinyl-N-oxide, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide. Preferred heteroaryl groups include pyridyl, pyrimidyl, quinolinyl, indolyl, pyrrolyl, furanyl, thienyl and imidazolyl, pyrazolyl, indazolyl, thiazolyl and benzothiazolyl. In certain embodiments, each heteroaryl is selected from pyridyl, pyrimidinyl, pyridazinyl, pyrazinyl, imidazolyl, isoxazolyl, pyrazolyl, oxazolyl, thiazolyl, furanyl, thienyl, pyrrolyl, oxadiazolyl, thiadiazolyl, triazolyl, tetrazolyl, isothiazolyl, pyridinyl-N-oxide, pyrrolyl N-oxide, pyrimidinyl N-oxide, pyridazinyl N-oxide, pyrazinyl N-oxide, imidazolyl N-oxide, isoxazolyl N-oxide, oxazolyl N-oxide, thiazolyl N-oxide, pyrrolyl N-oxide, oxadiazolyl N-oxide, thiadiazolyl N-oxide, triazolyl N-oxide, and tetrazolyl N-oxide. Preferred heteroaryl groups include pyridyl, pyrimidyl, quinolinyl, indolyl, pyrrolyl, furanyl, thienyl, imidazolyl, pyrazolyl, indazolyl, thiazolyl and benzothiazolyl. The heteroaryl groups herein are unsubstituted or, when specified as “optionally substituted”, can unless stated otherwise be substituted in one or more substitutable positions with various groups, as indicated.

The term “ring system” encompasses monocycles, as well as fused and/or bridged polycycles.

The term “oxo” means a doubly bonded oxygen, sometimes designated as ═O or for example in describing a carbonyl “C(O)” may be used to show an oxo substituted carbon.

The term “substituted.” when used to modify a specified group or radical, means that one or more hydrogen atoms of the specified group or radical are each, independently of one another, replaced with the same or different substituent groups as defined below, unless specified otherwise.

As used herein, the phrase “pharmaceutically acceptable salt” refers to both pharmaceutically acceptable acid and base addition salts and solvates. Such pharmaceutically acceptable salts include salts of acids such as hydrochloric, phosphoric, hydrobromic, sulfuric, sulfinic, formic, toluenesulfonic, methanesulfonic, nitric, benzoic, citric, tartaric, maleic, hydroiodic, alkanoic such as acetic, HOOC—(CH₂)ₙ—COOH where n is 0-4, and the like. Non-toxic pharmaceutical base addition salts include salts of bases such as sodium, potassium, calcium, ammonium, and the like. Those skilled in the art will recognize a wide variety of non-toxic pharmaceutically acceptable addition salts.

One of ordinary skill in the art of medicinal chemistry also will appreciate that the disclosed structures are intended to include isotopically enriched forms of the present compounds. As used herein “isotopes” includes those atoms having the same atomic number but different mass numbers. As is known to those of skill in the art, certain atoms, such as hydrogen occur in different isotopic forms. For example, hydrogen includes three isotopic forms, protium, deuterium and tritium. As will be apparent to those of skill in the art upon consideration of the present compounds, certain compounds can be enriched at a given position with a particular isotope of the atom at that position. For example, compounds having a fluorine atom, may be synthesized in a form enriched in the radioactive fluorine isotope ¹⁸F. Similarly, compounds may be enriched in the heavy isotopes of hydrogen: deuterium and tritium; and similarly can be enriched in a radioactive isotope of carbon, such as ¹³C. Such isotopic variant compounds undergo different metabolic pathways and can be useful, for example, in studying the ubiquitination pathway and its role in disease. Of course, in certain embodiments, the compound has substantially the same isotopic character as naturally-occurring materials.

One of ordinary skill in the art of chemistry will also appreciate that the disclosed structures, unless otherwise indicated are intended to include all possible stereoisomers of the claimed molecule, including mixtures of certain or all stereoisomers. However, compounds drawn with certain stereochemistry at one or more stereocenters are intended to have the indicated stereochemistry. Compounds and stereocenters drawn with ambiguous stereochemistry are meant to convey any stereoisomer or mixture thereof, e.g., a racemic mixture of compounds or a purified subset of stereoisomers.

As used herein, the terms “individual,” “patient,” or “subject” are used interchangeably, refers to any animal, including mammals, preferably humans.

As used herein, the phrase “therapeutically effective amount” or “effective amount” refers to the amount of active compound or pharmaceutical agent that elicits the biological or medicinal response that is being sought in a tissue, system, animal, individual or human by a researcher, veterinarian, medical doctor or other clinician.

In certain embodiments, an effective amount can be an amount suitable for
(i) inhibiting the progression the disease;
(ii) prophylactic use for example, preventing or limiting development of a disease, condition or disorder in an individual who may be predisposed or otherwise at risk to the disease, condition or disorder but does not yet experience or display the pathology or symptomatology of the disease;
(iii) inhibiting the disease; for example, inhibiting a disease, condition or disorder in an individual who is experiencing or displaying the pathology or symptomatology of the disease, condition or disorder;
(iv) ameliorating the referenced disease state, for example, ameliorating a disease, condition or disorder in an individual who is experiencing or displaying the pathology or symptomatology of the disease, condition or disorder (i.e., reversing or improving the pathology and/or symptomatology) such as decreasing the severity of disease; or
(v) eliciting the referenced biological effect.

As used here, the terms “treatment” and “treating” mean (i) ameliorating the referenced disease state, condition, or disorder (or a symptom thereof), such as, for example, ameliorating a disease, condition or disorder in an individual who is experiencing or displaying the pathology or symptomatology of the disease, condition or disorder (i.e., reversing or improving the pathology and/or symptomatology) such as decreasing the severity of disease or symptom thereof, or inhibiting the progression of disease; or (ii) eliciting the referenced biological effect (e.g., inhibiting inflammasome formation or function, or inhibition of IL-1β).

### Methods of Preparation

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any of the means known in the art, including chromatographic means, such as HPLC, preparative thin layer chromatography, flash column chromatography and ion exchange chromatography. Any suitable stationary phase can be used, including normal and reversed phases as well as ionic resins. Most typically the disclosed compounds are purified via silica gel and/or alumina chromatography. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry,” Plenum Press, London and New York 1973, in T. W. Greene and P. G. M. Wuts, “Protective Groups in Organic Synthesis,” Third edition, Wiley, New York 1999, in “The Peptides”; Volume 3 (editors: E. Gross and J. Meienhofer), Academic Press, London and New York 1981, in “Methoden der organischen Chemie,” Houben-Weyl, 4.sup.th edition, Vol. 15/l. Georg Thieme Verlag, Stuttgart 1974, in H.-D. Jakubke and H. Jescheit, “Aminosauren, Peptide, Proteine,” Verlag Chemie, Weinheim, Deerfield Beach, and Basel 1982, and/or in Jochen Lehmann, “Chemie der Kohlenhydrate: Monosaccharide and Derivate,” Georg Thieme Verlag, Stuttgart 1974. The protecting groups may be removed at a convenient subsequent stage using methods known from the art.

A “leaving group” as used herein refers to a moiety of a reactant (e.g., the alkylhalogenide of the disclosure) that is displaced from the first reactant in the chemical reaction. A comprehensive list of suitable leaving groups can be found in J. March, Advanced Organic Chemistry, John Wiley and Sons, N.Y. (2013). Examples of suitable leaving groups include, but are not limited to, halogen (such as Cl or Br), acetoxy, and sulfonyloxy groups (such as methyl sulfonyloxy, trifluoromethylsulfonyloxy (“triflate”), p-toluenesulfonyloxy (“tosylate”)).

The compounds disclosed herein can be made using procedures familiar to the person of ordinary skill in the art and as described herein, for example in Schemes 1. One of skill in the art can adapt the reaction sequences of schemes and examples as provided herein to fit the desired target molecule. Of course, in certain situations one of skill in the art will use different reagents to affect one or more of the individual steps or to use protected versions of certain of the substituents. Additionally, one skilled in the art would recognize that compounds of the disclosure can be synthesized using different routes altogether. For example, the person of ordinary skill in the art may adapt the procedures described herein and/or other procedures familiar to the person of ordinary skill in the art to make the compounds described herein.

### EXAMPLES

### General Synthesis Procedures

All reactions were conducted in oven-dried glassware under an inert atmosphere of argon using magnetic stirring. Thin layer chromatography (TLC) was performed on EMD silica gel 60 F254 plates and visualized under UV light. Column chromatography was performed on a Reveleris™ automated chromatography system. ¹H NMR spectra were recorded on a Varian Mercury (400 MHz) spectrometer. Chemical shifts are reported in ppm from tetramethylsilane with the solvent resonance as an internal standard (DMSO-d6: 2.49 ppm). Data are reported as follows: chemical shift, multiplicity (s=singlet, d=doublet, t=triplet, q=quartet, br=broad, m=multiplet), coupling constants (Hz), and integration. Low resolution mass spectrometry was performed on a Thermo Finnigan LXQ ion trap mass spectrometer equipped with an ESI source, which was used in the positive ion mode.

Compounds were synthesized using Schemes 2 and 3 as the key routes. A facile one-pot Gewald reaction (Scheme 1) was used to synthesize most of our desired 2-aminothiophene starting materials, III, by reacting cyanoacetamide, malonitrile or a cyanoacetyl derivative possessing an electron withdrawing group (EWG) as shown in compounds I, with an α-methylene ketone II and elemental sulfur in DMF solvent at 60° C. using imidazole as a catalyst (yields can go up to ˜90%). Scheme 2 will be used to synthesize the target products by reacting the 2-amino thiophenes (III) (obtained from Scheme 1) with the appropriate acid chlorides IV in the presence of base to obtain the target compounds (V. Synthesized compounds will be separated and purified by efficient flash chromatography using the Revelrys automated system, and the compound structures will be elucidation by physical methods: melting point, mass spectrometry (MS), infrared (IR) spectroscopy and high resolution nuclear magnetic resonance (NMR). Compound identity and purity will also be by elemental (C, H and N) analysis through Atlantic Labs (Norcross, GA) and HPLC, and if necessary high-resolution MS (HRMS) data. Synthesized compounds will be separated and purified by efficient automated flash chromatography using the Revelrys system and HPLC; and compound structures will be determined by physical methods: melting point, mass spectrometry (MS), infrared (IR) spectroscopy and high-resolution NMR and high-resolution mass spectrometry (HRMS). Determination of identity and purity will be done by elemental analysis, with ≥95% purity.

As an alternative to using acid chlorides for Scheme 2, the acids can be used to form the amides through carbodiimide coupling. To do that, 3-(ethyliminomethyleneamino)-N,N-dimethylpropan-1-amine (EDC), HCl (1.20 equiv.) and N,N-diisopropylethylamine (2.5 equiv.) will be added to a solution of the amine III (1 equiv.). acid (1 equiv.) and 1-hydroxybenztriazole (HOBt) (1.2 equiv.) in dimethylformamide (DMF) solvent (10 ml/1 mmol) and stirred for 24 hours at room temperature under nitrogen. The solvent was removed in vacuo and the residue purified by column chromatography on silica gel. It should also be noted that many of the aminothiophene starting materials (III) and analogs such as furan and pyrrole core templates are commercially available as starting materials to react with acid chlorides IV to shorten the synthetic route. In situations where the carbodiimide coupling is not efficient, we will synthesize the acid chlorides from available carboxylic acids by reaction with thionyl chloride (SOCl₂) or oxalyl chloride (ClCOCOCl). There are thus multiple pathways to synthesize target compounds. Through the entire period, at least 5 mg each of 200-250 new compounds will be synthesized with purities of ≥95% for testing. We will scale up synthesis to gm level for in vivo studies, and to make modifications to improve potency, selectivity or ADMETox and PK properties in an iterative fashion. We will deal with stereochemistry, thus if compounds comprise racemates, we will do chiral chromatographic separation, or chiral synthesis to obtain the pure enantiomers or diasteroisomers for biological studies.

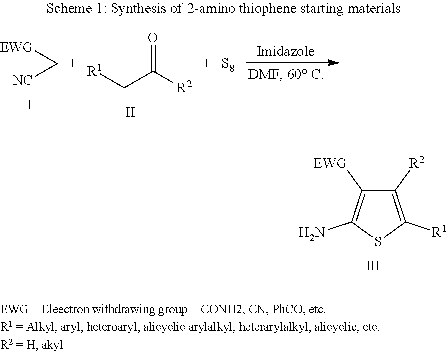

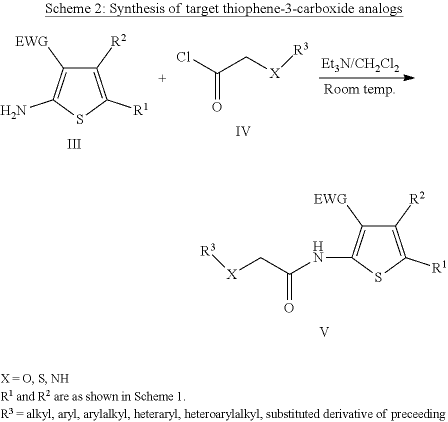

### Generic Synthesis Procedure

Amine (1 equiv), acid chloride (1.5 equiv), and triethylamine (2.5 equiv) were dissolved in dichloromethane and the reaction stirred for 24 hours. The resulting solution was washed three times with 1M HCl and the organic layers were collected. After drying with anhydrous magnesium sulfate and evaporation of the solvent, the resulting residue was purified by column chromatography on silica gel.

### Example 1

### Synthesis of Compound 1

Compound 1 was synthesized using Scheme 3, by reacting 2-amino thiophene with phenoxyacetyl chloride in the presence of base trimethylamine (Et₃N), using dichloromethane (CH₂Cl₂) as solvent at room temperature, and working up to isolate Compound 1 in 80% yield as a white solid (m.pt. 161-162° C.).

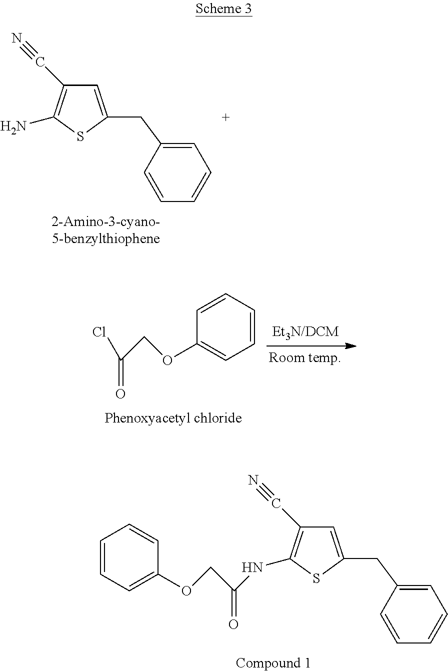 [SMILES: N#Cc1cc(Cc2ccccc2)sc1N ; O=C(Cl)COc1ccccc1 ; N#Cc1cc(Cc2ccccc2)sc1NC(=O)COc1ccccc1]

The above general procedure was employed to generate the following compounds:

N-(5-benzyl-3-cyanothiophen-2-yl)-2-phenoxyacetamide (1): ¹H NMR (400 MHz, DMSO-d₆) δ 4.35 (s, 2H), 4.72 (s, 2H), 6.83-6.90 (m, 3H), 7.05-7.25 (m, 8H), 11.05 (br s, 1H); ESI-MS m/z calcd for C₂₀H₁₆N₂O₂S [M +Na]⁺ 371.09, found 371.24.

N-(5-benzyl-3-cyanothiophen-2-yl)-2-phenoxypropanamide (2): ¹H NMR (400 MHz, DMSO-d₆) δ 1.48 (d, 3H, J=6.8 Hz), 4.37 (s, 2H), 4.72 (q, 1H, J=6.8 Hz), 6.93-7.01 (m, 3H), 7.10-7.25 (m, 8H), 11.55 (br s, 1H); ESI-MS m/z calcd for C₂₁H₁₈N₂O₂S [M+H]⁺ 363.11, found 363.25.

N-(5-benzyl-3-cyanothiophen-2-yl)-2-(m-tolyloxy)acetamide (3): ¹H NMR (400 MHz, DMSO-d₆) δ 2.22 (s, 3H), 4.39 (s, 2H), 4.76 (s, 2H), 6.72-6.80 (m, 3H), 7.17-7.28 (m, 7H), 11.50 (br s, 1H); ESI-MS m/z calcd for C₂₁H₁₈N₂O₂S [M+Na]⁺ 385.11, found 385.29.

5-benzyl-2-(2-(2,6-dimethylphenoxy)acetamido)-4-methylthiophene-3-carboxamide (4): ¹H NMR (400 MHz, DMSO-d₆) δ 2.05 (s, 6H), 2.12 (s, 3H), 4.35 (s, 2H), 4.53 (s, 2H), 7.05-7.28 (m, 10H), 11.45 (br s, 1H); ESI-MS m/z calcd for C₂₃H₂₄N₂O₃S [M−H]⁻ 407.14, found 407.32.

N-(5-benzyl-3-cyanothiophen-2-yl)benzofuran-2-carboxamide (5): ¹H NMR (400 MHz, DMSO-d₆) δ 4.35 (s, 2H), 7.18-7.28 (m, 7H), 7.49-7.59 (m, 4H), 11.10 (br s, 1H); ESI-MS m/z calcd for C₂₁H₁₄N₂O₂S [M−H]⁻ 357.08, found 357.09.

N-(5-benzyl-3-cyanothiophen-2-yl)-2-(3-(trifluoromethyl)phenoxy)acetamide (6): ¹H NMR (400 MHz, DMSO-d₆) δ 4.35 (s, 2H), 4.72 (s, 2H), 6.92-6.96 (m, 1H), 7.15-7.28 (m, 9H), 11.45 (br s, 1H); ESI-MS m/z calcd for C₂₁H₁₅N₂F₃O₂S [M−H]⁻ 415.08, found 415.19.

N-(5-benzyl-3-cyanothiophen-2-yl)-2-methyl-2-phenoxypropanamide (7): ¹H NMR (400 MHz, DMSO-d₆) δ 1.51 (s, 6H), 4.40 (s, 2H), 6.90-7.01 (m, 3H), 7.17-7.30 (m, 8H), 11.60 (br s, 1H); ESI-MS m/z calcd for C₂₂H₂₀N₂O₂S [M−H]⁻ 375.12, found 375.30.

5-benzyl-4-methyl-2-(2-phenoxypropanamido)thiophene-3-carboxamide (10): ¹H NMR (400 MHz, DMSO-d₆) δ 1.47 (d, 3H, J=6.8 Hz), 2.05 (s, 3H), 4.35 (s, 2H), 4.70 (q, 1H, J=6.8 Hz), 6.84-6.95 (m, 3H), 7.10-7.23 (m, 9H), 11.45 (br s, 1H); ESI-MS m/z calcd for C₂₂H₂₂N₂O₃S [M+Na]⁺ 417.14, found 417.33.

### Example 2

The compounds shown below in Table 1 can be prepared essentially according to procedures known to those of skill in the art in view of Scheme 1.

TABLE 1

| Comp. | | |
|---|---|---|
| No. | Name | Structure |
| 1 | N-(5-benzyl-3-cyanothiophen-2-yl)-2- phenoxyacetamide | 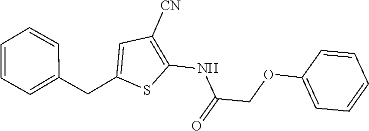 [SMILES: N#Cc1cc(Cc2ccccc2)sc1NC(=O)COc1ccccc1] |
| 2 | N-(5-benzyl-3-cyanothiophen-2-yl)-2- phenoxypropanamide | 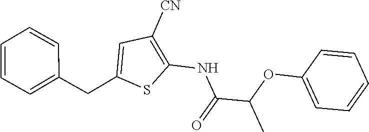 [SMILES: CC(Oc1ccccc1)C(=O)Nc1sc(Cc2ccccc2)cc1C#N] |
| 3 | N-(5-benzyl-3-cyanothiophen-2-yl)-2-(m- tolyloxy)acetamide | 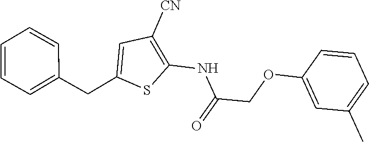 [SMILES: Cc1cccc(OCC(=O)Nc2sc(Cc3ccccc3)cc2C#N)c1] |
| 4 | 5-benzyl-2-(2-(2,6- dimethylphenoxy)acetamido)-4- methylthiophene-3-carboxamide | 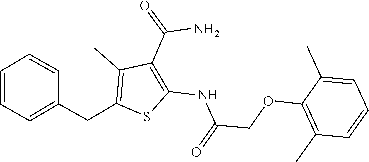 [SMILES: Cc1cccc(C)c1OCC(=O)Nc1sc(Cc2ccccc2)c(C)c1C(N)=O] |
| 5 | N-(5-benzyl-3-cyanothiophen-2- yl)benzofuran-2-carboxamide | 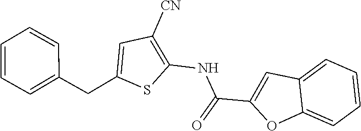 [SMILES: N#Cc1cc(Cc2ccccc2)sc1NC(=O)c1cc2ccccc2o1] |
| 6 | N-(5-benzyl-3-cyanothiophen-2-yl)-2-(3- (trifluoromethyl)phenoxy)acetamide | 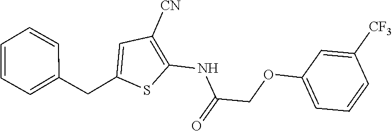 [SMILES: N#Cc1cc(Cc2ccccc2)sc1NC(=O)COc1cccc(C(F)(F)F)c1] |
| 7 | N-(5-benzyl-3-cyanothiophen-2-yl)-2- methyl-2-phenoxypropanamide | 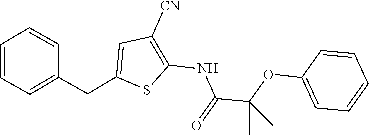 [SMILES: CC(C)(Oc1ccccc1)C(=O)Nc1sc(Cc2ccccc2)cc1C#N] |
| 8 | N-(5-benzyl-3-cyanothiophen-2-yl)-2-(4- (trifluoromethoxy)phenoxy)acetamide | 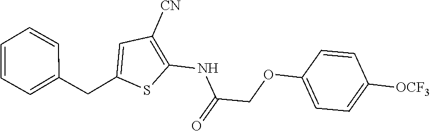 [SMILES: N#Cc1cc(Cc2ccccc2)sc1NC(=O)COc1ccc(OC(F)(F)F)cc1] |
| 9 | N-(5-benzyl-3-cyanothiophen-2-yl)-2-(4- chlorophenoxy)acetamide | 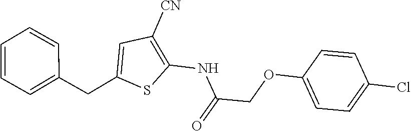 [SMILES: N#Cc1cc(Cc2ccccc2)sc1NC(=O)COc1ccc(Cl)cc1] |
| 10 | 5-benzyl-4-methyl-2-(2- phenoxypropanamido)thiophene-3- carboxamide | 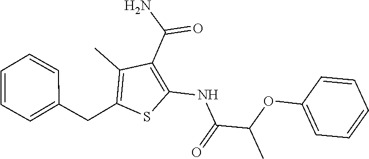 [SMILES: Cc1c(Cc2ccccc2)sc(NC(=O)C(C)Oc2ccccc2)c1C(N)=O] |

### Example 3: Inhibition of STAT3 Phosphorylation in GMB Cells

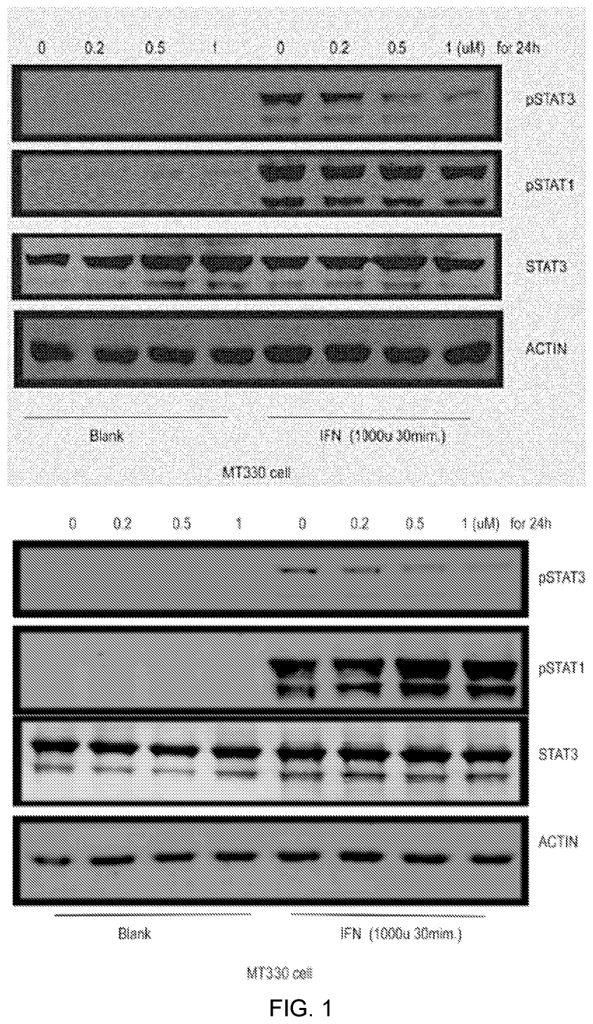

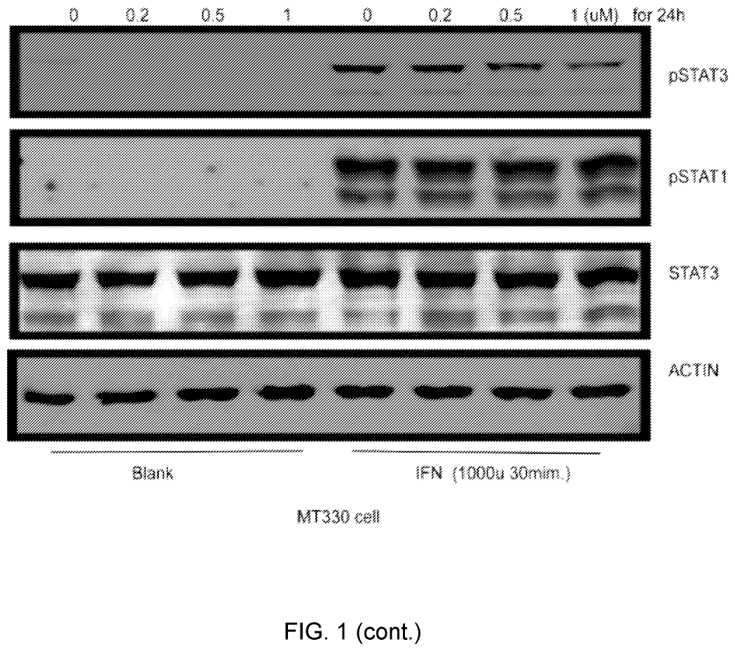

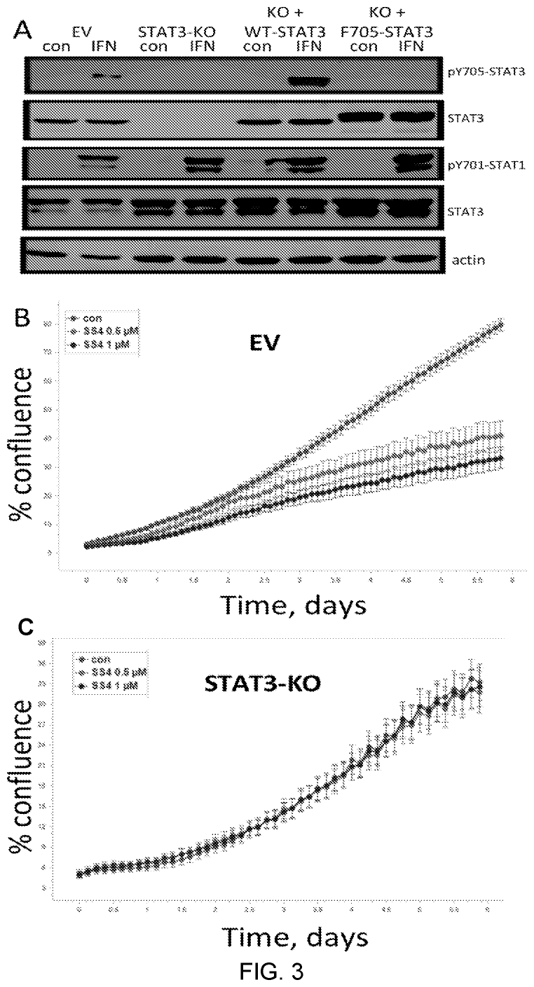

In assessing the potential of this new generation of STAT3 inhibitors for further development, we tested their ability to selectively inhibit STAT3 phosphorylation relative to STAT1 by western blotting. As FIG. 3 shows, when STAT3 and STAT1 phosphorylation was induced by IFN, the compounds, 1, 5 and 7 (Table 1) were able to potently block STAT3 phosphorylation at submicromolar levels but had no effect on STAT1 phosphorylation (FIG. 1). The mechanism of how Compound 1 inhibits STAT3 phosphorylation is currently being investigated but with the strong data on inhibition of STAT3 phosphorylation relative to STAT1, the nitriles represented by Compound 1, DP3 and DP5 emerged as attractive powerful new generation of STAT3 inhibitors.

### Example 4: Antiproliferative Activity

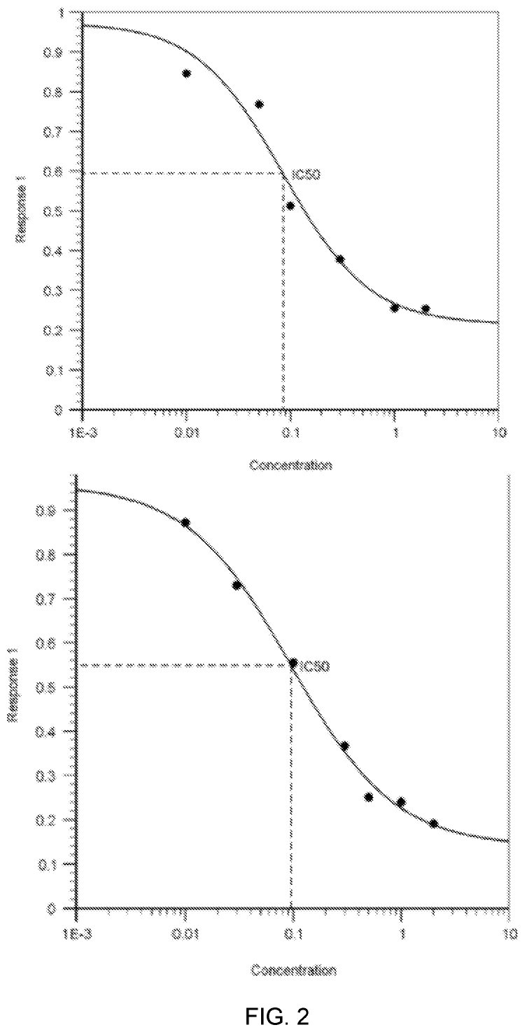

The ability of Compound 1 against proliferation of GBM cell lines, and its dependence on STAT3 as the target was tested. The results show that Compound 1 potently of both MT300 and LN299 GBM cells proliferation, with impressive IC₅₀ values of 85 nM and 95 and, respectively (FIG. 2).

### Example 5: Gene Knockout Studies

To further characterize that STAT3 is the target of Compound 1 in killing glioma cells, STAT3-knockout (STAT3-KO) GBM cell lines were developed by lentiviral transduction. The STAT3 ablation in the STAT3-KO cells was determined by immunoblotting with STAT3 while STAT1 expression was unaffected (FIG. 3A). Thus, whereas IFNα treatment induced robust STAT3 and STAT1 activation in empty vector (EV) transduced MT300 cells, STAT3 activation was absent in the STAT3-KO cells (FIG. 3A). More importantly, while the parental GBM cells were sensitive to the antiproliferative effect of Compound 1 (FIG. 3B), STAT3-KO GBM cells were resistant to Compound 1 (FIG. 3C). Thus, Compound 1 can kill GBM cells in a STAT3 dependent fashion at nM levels.

### Example 6: Cancer Cell Specification STAT3 Inhibition

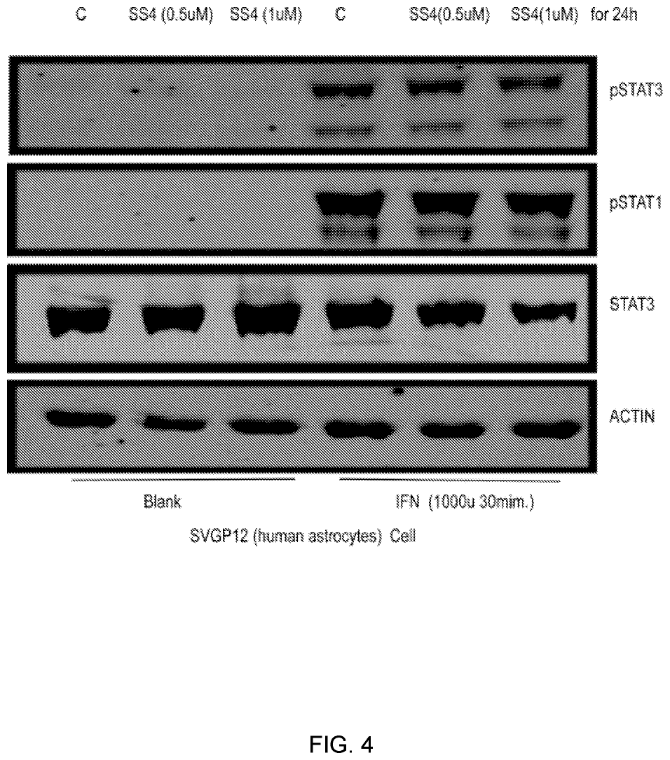

Importantly, Compound 1 has also demonstrated selectivity towards cancer cells relative to normal cells, by not inhibiting STAT3 phosphorylation in normal human astrocytes, in which it was unable to inhibit IFN-induced STAT3 activation (FIG. 4). This is unlike the case with MT300 GBM cells in which Compound 1 potently inhibited.

### Example 7: Isothermal Titration Calorimetry Studies of STAT3 Binding

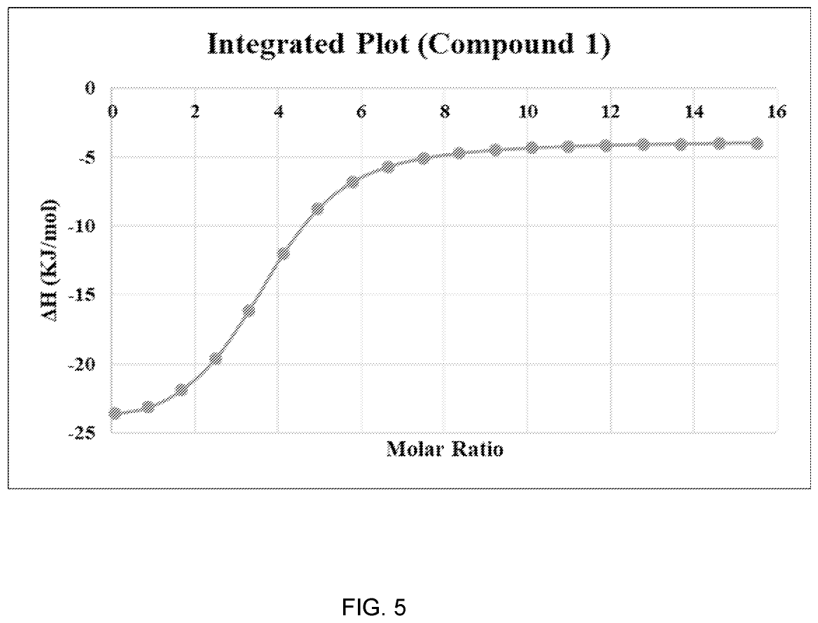

ITC studies was performed, as previously described (Alonso-Valenteen, et al. Nucleic acids research 2019, 47, 11020-11043; Brotherton-Pleiss et al., J. J Med Chem. 2021 Jan. 14; 64(1):695-710). The binding isotherm from the integrated thermogram fit using the one-site model in the PEAQ-ITC software generated from the titration of Compound 1 into purified STAT3 show K_{D} of 880 nM. The signature plot show the thermodynamics parameters for the titration reveals ΔH=−21.1 kJ/mol, ΔG=−34.6 kJ/mol, and −TΔS=−13.2 kJ/mol. Results show Compound 1 compound directly binds to STAT3 with high affinity (FIG. 5 and Table 2).

TABLE 2

| | | | ΔH | ΔG | −TΔS |
|---|---|---|---|---|---|
| Compound | N | K_{D} (M) | (KJ/mol) | (KJ/mol) | (KJ/mol) |
| 1 | 3.52 | 8.80E−07 | −21.1 | −34.6 | −13.2 |

### Example 8: Evaluation for In Vitro Biological Activity and Selectivity, and Investigation of Mechanisms

### In Vivo Antitumor Efficacy

The prioritized compounds will be evaluated in the in vivo studies here and compared to Compound 1 and WP1066 (a current STAT3 inhibitor in GBM clinical trials). It is anticipated that we will test 4-5 new optimized Compound 1 analogs for in vivo efficacy over the duration of the grant proposal. Based on our in vitro and in vivo findings, we will test the efficacies of Compound 1, and select, prioritized analogs (at least one per year), and will compare to WP1066 on GBM cancer growth in vivo. We will also investigate the mechanism(s) of cancer suppression by this new class of STAT3 inhibitors.

### Preliminary Studies: STAT3 Inhibitors Exhibit In Vivo Antitumor Effects

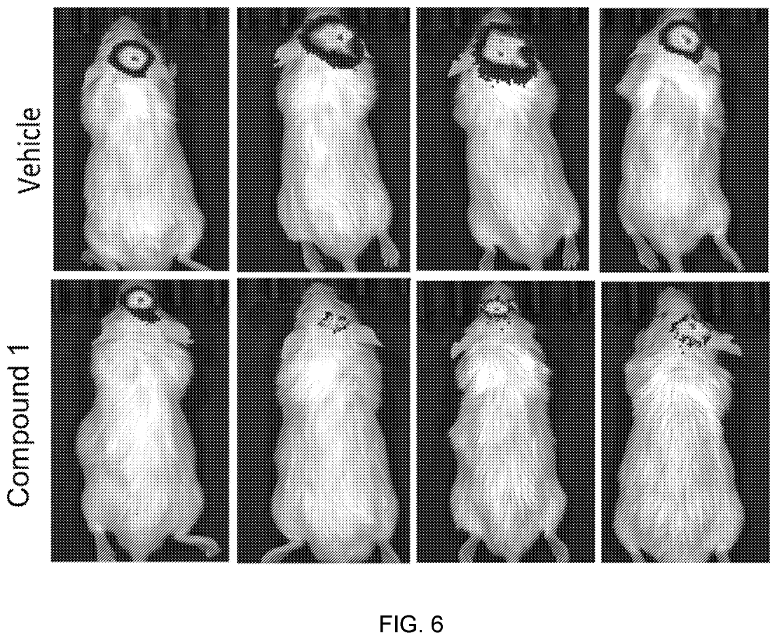

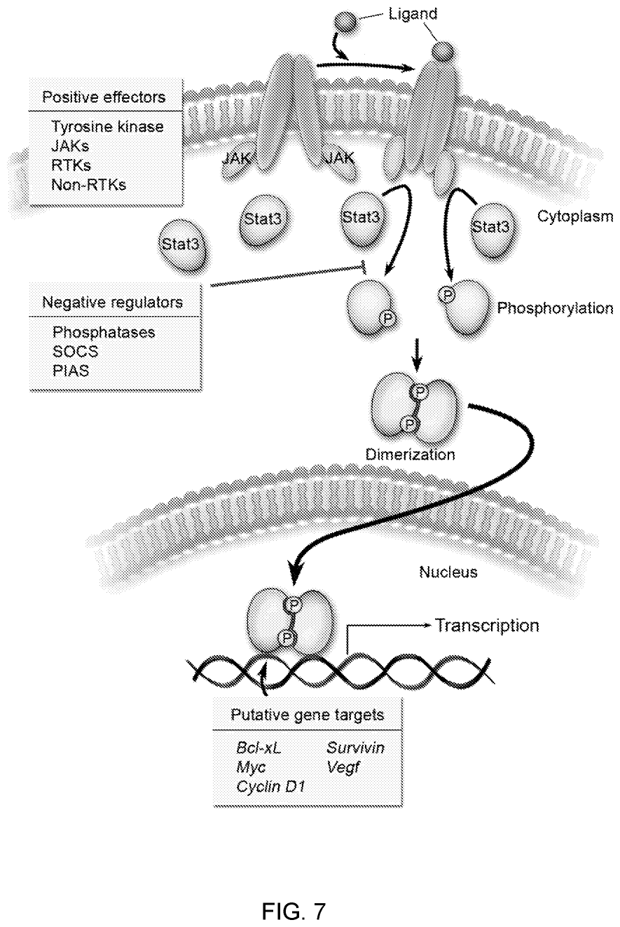

To generate data to support in vivo testing of Compound 1 effects on GBM tumors we recently performed the following initial proof-of-concept experiments using MT330 glioma xenografts. In brief, 10⁶ luciferase-expressing MT330 GBM cells were injected orthotopically into the brains of female NGS mice. Once tumor formation was verified by live animal bioluminescent imaging (BLI, 10-14 days after injection), 10 mice were randomly assigned into two groups (5 mice per group) and injected into the peritoneum three times per week with: a) Compound 1 in DMSO (20 mg/Kg), or b) vehicle control (DMSO) (FIG. 6). Tumor growth was monitored by BLI two times per week as we have previously described. Compound 1 treatment markedly reduced orthotopic GBM tumor xenografts (FIG. 6), indicating it has anti-GBM activity in vivo, and shows that it penetrates the blood-brain barrier.

### Study Design and Analysis: GMB Tumorigenicity and Sensitivity to STAT3 Inhibitors

We will determine which route of delivery (intraperitoneal, intravenous or oral gavage) will increase the ability of Compound 1 or analogs to cross the BBB which is a major impediment to GBM therapy. Thus, groups of 5 male and female mice each will be subjected to the 3 delivery routes of Compound 1 or analog (5, 10 or 20 mg/Kg) and after 1 day euthanized and the levels of Compound 1 in the brain, liver, spleen, blood and other organs will be determined by mass spectrometry as we previously performed. Once we identify which route of administration is best for the STAT3 inhibitors to cross the BBB, we will determine the effects of this inhibitor in the orthotopic microenvironment for GBM by intracranial injection of tumor cells. In brief, luciferase-expressing GSCs (3×10⁴) will be injected stereotactically into the brains of female NSG mice, and mice subjected to live animal BLI at weekly intervals.

Once tumors are detected (˜1-2 weeks post-injection), mice will be randomly divided into 8 groups and treated 5 days per week for two weeks: 1) vehicle treatment alone; 2) WP1066 at 20 mg/Kg; 3) Compound 1 at 20 mg/Kg; 4) optimized Compound 1 analog at 20 mg/ml; 5) TMZ treatment at 50 mg/Kg; 6) the combination of WP1066 and TMZ; 7) the combination of Compound 1 with TMZ, and 8) the combination of Compound 1 analog with TMZ. Combination Index (CI) values will be calculated using the Chou-Talalay method. CI values of <1 will indicate synergy, while values of 1 or >1, will indicate additivity or antagonism, respectively.

NSG mice will be injected with D-luciferin and subjected to BLI animal imaging weekly to quantify bioluminescence. Tumor growth will be assessed by live animal imaging (see FIG. 12) and animal survival within the study groups by Kaplan-Meier plots. Tumor growth will then be compared among the groups using a Wilcoxon rank-sum test. It is expected that with 10 mice in each group we will have at least 80% power to detect a 50% reduction in volume with type I error control at α=0.05. This general approach will assess the effect of STAT3 inhibitors on GBM tumorigenicity, and on the chemosensitivity of GBM cells. After sacrifice, tumors will be removed, formalin fixed and embedded in paraffin, or they will be snap frozen for the mechanistic studies. Tumors will also be subjected to histology (H&E staining) and immunohistology, and RNA and protein will be extraction to determine effects on expression of GBM prognostic markers (GFAP, S100B, Olig2, Map2 and Syn). Tumor tissues will also be stained for apoptosis by TUNEL assays and caspase and PARP cleavage, and DNA damage (γH2Ax) as we performed previously.

### Example 9: Biological Screening of Compounds

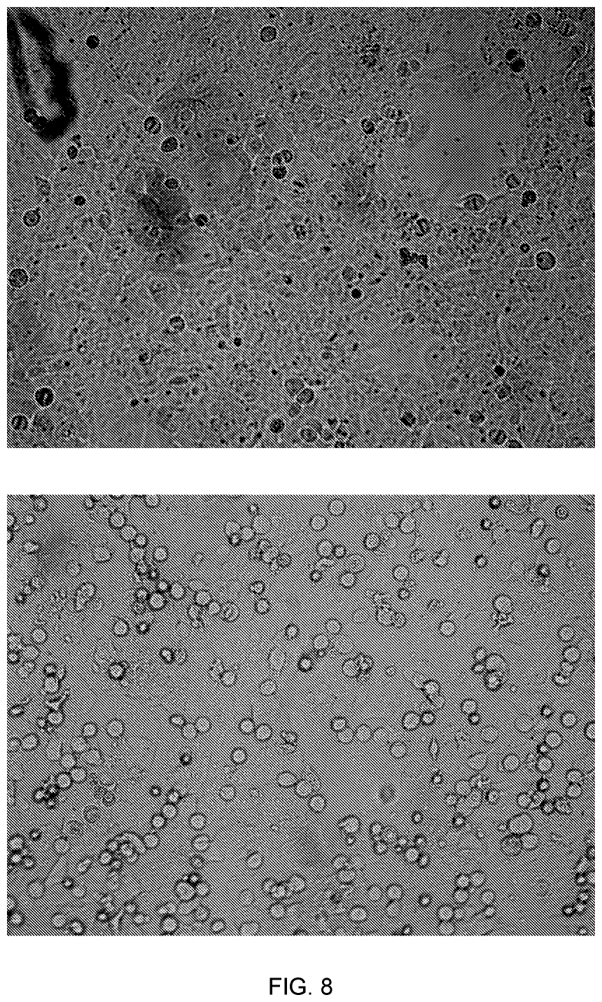

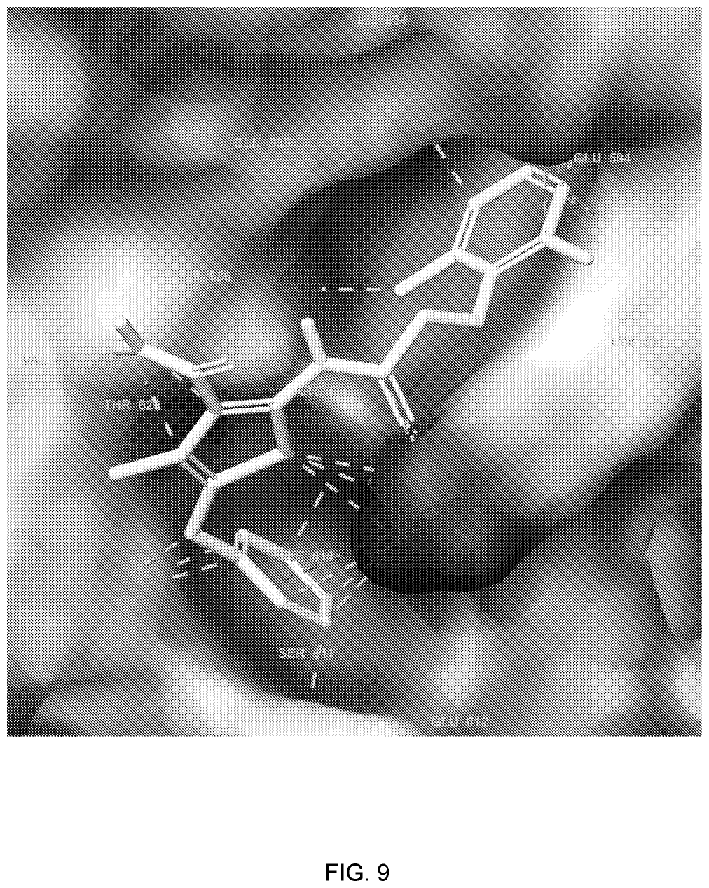

MTS assay was used to determine the cell viability for all compounds tested. Computational docking scores were used to evaluate potential for STAT 3 inhibition (FIG. 9 and Table 3). MCF7 breast cancer cells were grown until 80-90% confluence. Cells were then transferred to a Costar 96-well plate with about 3000 cells per well. Cells were incubated for 24 hours. After incubation cells were treated with appropriate compounds and concentration for 72 hours. Twenty microliters of MTS solution was added to each well and incubated for 2 hours. The plate was then analyzed using fluorescent measurement at 360/40 excitation and 460/40 emission. The results of this screening is shown in FIG. 8 and Table 3, below:

TABLE 3

| Compound | Docking score | Percent inhibition |
|---|---|---|
| 7 | −2.78 | 23.33 |
| 5 | −2.46 | 65.67 |
| 1 | −2.46 | 28.06 |
| 6 | −1.74 | 51.00 |

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

Moreover, there are disclosed the following embodiments:

Embodiment 1. A compound having the structural formula:

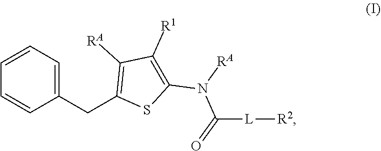

or a pharmaceutically acceptable salt thereof,
wherein:
each R^{A} is independently H or methyl;
R¹ is —CN or —C(O)—N(R^{A})₂;
R² is aryl or heteroaryl, wherein the aryl or heteroaryl is monocyclic or bicyclic and comprised of 5-10 ring members, and optionally substituted with 1-4 R^{B}, wherein R^{B} is C₁-C₂ alkyl, fluoro, chloro, or —O—(C₁-C₂ alkyl), wherein each alkyl is optionally fluorinated.
L is absent or —C(R^{A})₂—O—.

Embodiment 2. The compound of embodiment 1, wherein R¹ is —CN or —C(O)—NH₂.

Embodiment 3. The compound of embodiment 1, wherein R¹ is —CN.

Embodiment 4. The compound of any of embodiments 1-3, wherein R² is phenyl or benzofuranyl.

Embodiment 5. The compound of embodiment 4, wherein R² is phenyl.

Embodiment 6. The compound of any of embodiments 1-5, wherein R² is unsubstituted.

Embodiment 7. The compound of any of embodiments 1-5, wherein R² is substituted with 1-2 R^{B}, wherein R^{B} is CH₃, CF₃, chloro, —O—CH₃, or —O—CF₃.

Embodiment 8. The compound of any of embodiments 1-7, wherein L is absent.

Embodiment 9. The compound of any of embodiments 1-7, wherein L is —C(R^{A})₂—O—.

Embodiment 10. The compound of embodiment 9, wherein L is —C(CH₃)₂—O— or —CH₂—O—.

Embodiment 11. The compound of embodiment 9, wherein L is —CH(CH₃)—O—.

Embodiment 12. The compound of any of embodiments 1-11, wherein the compound is not 5-benzyl-2-(2-(2-methoxyphenoxy)acetamido)-4-methylthiophene-3-carboxamide, N-[3-carbamoyl-5-benzyl-thien-2-yl]4-hydroxyphenoxyacetamide, or 5-benzyl-4-methyl-2-(2-phenoxyacetamido)thiophene-3-carboxamide.

Embodiment 13. The compound of any of embodiments 1-12, wherein the compound is selected from a group consisting of N-(5-benzyl-3-cyanothiophen-2-yl)-2-phenoxyacetamide, N-(5-benzyl-3-cyanothiophen-2-yl)-2-phenoxypropanamide, N-(5-benzyl-3-cyanothiophen-2-yl)-2-(m-tolyloxy)acetamide, 5-benzyl-2-(2-(2,6-dimethylphenoxy)acetamido)-4-methylthiophene-3-carboxamide, N-(5-benzyl-3-cyanothiophen-2-yl)benzofuran-2-carboxamide, N-(5-benzyl-3-cyanothiophen-2-yl)-2-(3-(trifluoromethyl)phenoxy)acetamide, N-(5-benzyl-3-cyanothiophen-2-yl)-2-methyl-2-phenoxypropanamide, N-(5-benzyl-3-cyanothiophen-2-yl)-2-(4-(trifluoromethoxy)phenoxy)acetamide, N-(5-benzyl-3-cyanothiophen-2-yl)-2-(4-chlorophenoxy)acetamide, and 5-benzyl-4-methyl-2-(2-phenoxypropanamido)thiophene-3-carboxamide.

Embodiment 14. A method of treating cancer in a subject in need thereof, comprising administering a therapeutically effect amount of a compound according to any of embodiments 1-13.

Embodiment 15. The method of embodiment 14, wherein the cancer is selected from breast cancer, glioblastoma multiforme brain cancer, colorectal cancer, lung cancer, pancreatic cancer, bladder cancer, and metastatic prostate cancer.

### REFERENCES

1. Akira, S.; Nishio, Y.; Inoue, M.; Wang, X.J.; Wei, S.; Matsusaka, T.; Yoshida, K.; Sudo, T.; Naruto, M.; Kishimoto, T. Molecular cloning of aprf, a novel ifn-stimulated gene factor 3 p91-related transcription factor involved in the gp130-mediated signaling pathway. Cell 1994, 77,63-71.
2. Baell, JB, Holloway, GA. New substructure filters for removal of pan assay interference compounds (PAINS) from screening libraries and for their exclusion in bioassays. J Med Chem 2010, 53, 2719-2740
3. Becker S.; Groner B.; Muller C. W. Three-dimensional structure of the STAT3beta homodimer bound to DNA. Nature 1998, 394, 145-15110.
4. Bowman, T.; Garcia, R.; Turkson, J.; Jove, R. Stats in oncogenesis. Oncogene 2000, 19, 2474-2488.
5. Brennan, C.W.; Verhaak, R.G.; McKenna, A.; Campos, B.; Noushmehr, H.; Salama, S.R.; Zheng, S.; Chakravarty, D.; Sanborn, J.Z.; Berman, S.H., et al. The somatic genomic landscape of glioblastoma. Cell 2013, 155, 462-477.
6. Brotherton-Pleiss C, Yue P, Zhu Y, Nakamura K, Chen W, Fu W, Kubota C, Chen J, Alonso-Valenteen F, Mikhael S, Medina-Kauwe L, Tius MA, Lopez-Tapia F, Turkson J. Discovery of novel azetidine amides as potent mmall-molecule STAT3 inhibitors. J Med Chem 2021, 64, 695-710.
7. Bu, L.L.; Yu, G.T.; Deng, W.W.; Mao, L.; Liu, J.F.; Ma, S.R.; Fan, T.F.; Hall, B.; Kulkarni, A.B.; Zhang, W.F., et al. Targeting stat3 signaling reduces immunosuppressive myeloid cells in head and neck squamous cell carcinoma. Oncoimmunology 2016. 5, e1130206.
8. Buolamwini, J.K., Raghavan, K., Pommier, Y., Kohn, K.W., Weinstein, J. N. Application of the electrotopological state index to QSAR analysis of flavone derivatives as HIV-1 integrase inhibitors. Pharm Res 1996, 13, 1891-1894.
9. Cancer Genome Atlas Research, N. Comprehensive genomic characterization defines human glioblastoma genes and core pathways. Nature 2008, 455, 1061-1068.
10. Castro, B, Dormoy, JR, Evin, G, Castro B. Peptide coupling reagents IV (1) benzotriazole N-oxytrisdimethylamino phosphonium hexafluorophosphate (B.O.P.) Tetrahedron Lett. 1975, 1219.
11. Chen Q, Buolamwini JK, Smith JC, Li A, Xu Q, Cheng X, Wei D. Impact of resistance mutations on inhibitor binding to HIV-1 integrase. J Chem Inf Model 2013, 53, 3297-307.
12. Chou, T. C. Talalay P. Quantitative analysis of dose-effect relationships: the combined effects of multiple drugs or enzyme inhibitors. Adv Enzyme Regul 1984, 22, 27-55.
13. Clarke, M.F.; Dick, J.E.; Dirks, P.B.; Eaves, C.J.; Jamieson, C.H.; Jones, D.L.; Visvader, J.; Weissman, I.L.; Wahl, G.M. Cancer stem cells—perspectives on current status and future directions: Aacr workshop on cancer stem cells. Cancer Res 2006, 66, 9339-9344.
14. Darnell, J.E., Jr.; Kerr, I.M.; Stark, G.R. Jak-stat pathways and transcriptional activation in response to ifns and other extracellular signaling proteins. Science 1994, 264, 1415-1421.
15. Donkor IO, Li H, Queener S.F. Synthesis and DHFR inhibitory activity of a series of 6-substituted2,4-diaminothieno[2,3-d]pyrimidines. Eur J Med Chem. 2003, 38, 605-611.
16. Du, Z.; Cai, C.; Sims, M.; Boop, F.A.; Davidoff, A.M.; Pfeffer, L.M. The effects of type i interferon on glioblastoma cancer stem cells. Biochem Biophys Res Commun 2017, 491, 343-348.
17. Du, Z.; Fan, M.; Kim, J.G.; Eckerle, D.; Lothstein, L.; Wei, L.; Pfeffer, L.M. Interferon-resistant daudi cell line with a stat2 defect is resistant to apoptosis induced by chemotherapeutic agents. J Biol Chem 2009, 284, 27808-27815.
18. Fan, M.; Pfeffer, S.R.; Lynch, H.T.; Cassidy, P.; Leachman, S.; Pfeffer, L.M.; Kopelovich, L. Altered transcriptome signature of phenotypically normal skin fibroblasts heterozygous for cdkn2a in familial melanoma: Relevance to early intervention. Oncotarget 2013, 4, 128-141.
19. Galli, R.; Binda, E.; Orfanelli, U.; Cipelletti, B.; Gritti, A.; De Vitis, S.; Fiocco, R.; Foroni, C.; Dimeco, F.; Vescovi, A. Isolation and characterization of tumorigenic, stem-like neural precursors from human glioblastoma. Cancer Res 2004, 64, 7011-7021.
20. Gallo, M.: Coutinho, F.J.; Vanner, R.J.; Gayden, T.; Mack, S.C.; Murison, A.; Remke, M.; Li, R.; Takayama, N.; Desai, K., et al. MII5 orchestrates a cancer self-renewal state by repressing the histone variant h3.3 and globally reorganizing chromatin. Cancer Cell 2015, 28, 715-729.
21. Gangemi, R.M.; Griffero, F.; Marubbi, D.; Perera, M.; Capra, M.C.; Malatesta, P.; Ravetti, G.L.; Zona, G.L.; Daga, A.; Corte, G. Sox2 silencing in glioblastoma tumor-initiating cells causes stop of proliferation and loss of tumorigenicity. Stem Cells 2009, 27, 40-48.
22. Ganguly, D.; Fan, M.; Yang, C.H.; Zbytek, B.; Finkelstein, D.; Roussel, M.F.; Pfeffer, L.M. The critical role that stat3 plays in glioma-initiating cells: Stat3 addiction in glioma. Oncotarget 2018, 9, 22095-22112.
23. Ganguly, D.; Sims, M.: Cai, C.; Fan, M.; Pfeffer, L.M. Chromatin remodeling factor brg 1 regulates stemness and chemosensitivity of glioma initiating cells. Stem Cells 2018, 36, 1804-1815.
24. Garner, J.M.; Ellison, D.W.; Finkelstein, D.; Ganguly, D.; Du, Z.; Sims, M.; Yang, C.H.; Interiano, R.B.; Davidoff, A.M.; Pfeffer, L.M. Molecular heterogeneity in a patient-derived glioblastoma xenoline is regulated by different cancer stem cell populations. PLOS One 2015, 10, e0125838.
25. Garner, J.M.; Fan, M.; Yang, C.H.; Du, Z.; Sims, M.; Davidoff, A.M.; Pfeffer, L.M. Constitutive activation of signal transducer and activator of transcription 3 (stat3) and nuclear factor kappab signaling in glioblastoma cancer stem cells regulates the notch pathway. J Biol Chem 2013, 288, 26167-26176.
26. Huang X-G, Liu J., Ren, J., Wang, T., Chen, W., Zeng, B.-B. A facile and practical one-pot synthesis of multisubstituted 2-aminothiophenes via imidazole-catalyzed Gewald reaction. Tetrahedron 2011, 67, 6202-6205.
27. Huntly, B.J.; Gilliland, D.G. Leukaemia stem cells and the evolution of cancer-stem-cell research. Nat Rev Cancer 2005, 5, 311-321.
28. Joo. K.M.; Kim, J.; Jin, J.; Kim, M.; Seol, H.J.; Muradov, J.; Yang, H.; Choi, Y.L.; Park, W.Y.; Kong, D.S., et al. Patient-specific orthotopic glioblastoma xenograft models recapitulate the histopathology and biology of human glioblastomas in situ. Cell Reports 2013, 3, 260-273.
29. Keir, S.T.; Maris, J.M.; Reynolds, C.P.; Kang, M.H.; Kolb, E.A.; Gorlick, R.; Lock, R.; Carol, H.; Morton, C.L.; Wu, J., et al. Initial testing (stage 1) of temozolomide by the pediatric preclinical testing program. Pediatric Blood & Cancer 2013, 60, 783-790.
30. Kumar H. Deokar, Hilaire Playa Barch, Buolamwini John K, Homology Modeling of Human Concentrative Nucleoside Transporters (hCNTs) and Validation by Virtual Screening and Experimental Testing to Identify Novel hCNT1 Inhibitors. Drug Des 2017, 6, 146 doi: 10.4172/2169-0138.1000146.
31. Li, R.; Hu, Z.: Sun, S.Y.; Chen, Z.G.; Owonikoko, T.K.; Sica, G.L.; Ramalingam, S.S.; Curran, W.J.; Khuri, F.R.; Deng, X. Niclosamide overcomes acquired resistance to erlotinib through suppression of stat3 in non-small cell lung cancer. Mol Cancer Ther 2013, 12, 2200-2212.
32. Lipinski CA, Lombardo F, Dominy BW, Feeney PJ. Experimental and computational approaches to estimate solubility and permeability in drug discovery and development settings. Adv Drug Deliv Rev 2001, 46, 3-26.
33. Lipinski CA. Drug-like properties and the causes of poor solubility and poor permeability. J. Pharmacol Toxico. Methods 2000, 44, 235-249.
34. Lopez-Tapia, F, Brotherton-Pleiss C, Yue P, Murakami H, Costa Araujo AC, Reis Dos Santos B, Ichinotsubo E, Rabkin A, Shah R, Lantz M, Chen S, Tius MA, Turkson J, Linker variation and structure-activity relationship analyses of carboxylic acid-based small molecule STAT3 inhibitors. ACS Med Chem Lett 2018, 9, 250-255.
35. Mangiola, A.; Lama, G.; Giannitelli, C.; De Bonis, P.; Anile, C.; Lauriola, L.; La Torre, G.; Sabatino, G.; Maira, G.; Jhanwar-Uniyal, M., et al. Stem cell marker nestin and c-jun nh2-terminal kinases in tumor and peritumor areas of glioblastoma multiforme: Possible prognostic implications. Clin Cancer Res 2007, 13, 6970-6977.
36. Mani, S.A.; Guo, W.; Liao, M.J.; Eaton, E.N.; Ayyanan, A.; Zhou, A.Y.; Brooks, M.; Reinhard, F.; Zhang, C.C.; Shipitsin, M., et al. The epithelial-mesenchymal transition generates cells with properties of stem cells. Cell 2008, 133, 704-715.
37. Marotta, L.L.; Polyak, K. Cancer stem cells: A model in the making. Curr Opin Genet Dev 2009, 19, 44-50.
38. Miklossy, G, Hilliard, TS, Turkson, J, Therapeutic modulators of STAT signalling for human diseases, Nature Rev Drug Discov 2013. 12, 611-629.
39. Park I.H., Li C. Characterization of molecular recognition of STAT3 SH2 domain inhibitors through molecular simulation. J Mol Recognit 2011, 24, 254-265.
40. Patel, A.P.; Tirosh, I.; Trombetta, J.J.; Shalek, A.K.; Gillespie, S.M.; Wakimoto, H.; Cahill, D.P.; Nahed, B.V.; Curry, W.T.; Martuza, R.L., et al. Single-cell rna-seq highlights intratumoral heterogeneity in primary glioblastoma. Science 2014, 344, 1396-1401.
41. Peng, Z.; Zhang, C.; Zhou, W.; Wu, C.; Zhang. Y. The stat3/nfil3 signaling axis-mediated chemotherapy resistance is reversed by raddeanin a via inducing apoptosis in choriocarcinoma cells. J Cell Physiol 2018, 233, 5370-5382.
42. Pfeffer, L.M.; Mullersman, J.E.; Pfeffer, S.R.; Murti, A.; Shi, W.; Yang, C.H. Stat3 as an adapter to couple phosphatidylinositol-3 kinase to the ifnar-1 chain of the type I IFN receptor. Science 1997, 276, 1418-1420.
43. Pfeffer, S.R.; Fan, M.; Du, Z.; Yang, C.H.; Pfeffer, L.M. Unphosphorylated stat3 regulates the antiproliferative, antiviral, and gene-inducing actions of type i interferons. Biochem Biophys Res Commun 2017, 490, 739-745.
44. Pollard, S.M.; Yoshikawa, K.; Clarke, I.D.; Danovi, D.; Stricker, S.; Russell, R.; Bayani, J.; Head, R.; Lee, M.; Bernstein, M., et al. Glioma stem cell lines expanded in adherent culture have tumor-specific phenotypes and are suitable for chemical and genetic screens. Cell Stem Cell 2009, 4, 568-580.
45. Roeser, J.C.; Leach, S.D.; McAllister, F. Emerging strategies for cancer immunoprevention. Oncogene 2015, 34, 6029-6039.
46. Sausville, E.A.; Burger, A.M. Contributions of human tumor xenografts to anticancer drug development. Cancer Res 2006, 66, 3351-3354, discussion 3354.
47. Sen, M.; Joyce. S.; Panahandeh, M.; Li, C.; Thomas, S.M.; Maxwell, J.; Wang, L.; Gooding, W.E.; Johnson, D.E.; Grandis, J.R. Targeting stat3 abrogates egfr inhibitor resistance in cancer. Clin Cancer Res 2012, 18, 4986-4996.
48. Sen, M.; Thomas, S.M.; Kim, S.; Yeh, J.I.; Ferris, R.L.; Johnson, J.T.; Duvvuri, U.; Lee, J.; Sahu, N.; Joyce, S., et al. First-in-human trial of a stat3 decoy oligonucleotide in head and neck tumors: Implications for cancer therapy. Cancer Discov 2012. 2. 694-705.
49. Shahani, VM, Yue, P, Haftchenary, S. Zhao, W, Lukkarila, JL, Zhang, X, Ball, D, Nona, C, Gunning PT, Turkson J, Identification of purine-scaffold small-molecule inhibitors of STAT3 activation by QSAR studies, ACS Med Chem Lett 2011, 2, 79-84.
50. Sharma H, Cheng X, Buolamwini JK. Homology model-guided 3D-QSAR studies of HIV-1 integrase inhibitors. J Chem Inf Mode. 2012, 52, 515-544.
51. Sheehan JC, Cruickshank PA, Boshart GL. Convenient synthesis of water-soluble carbodiimides. J Org Chem 1961, 26, 2525.
52. Siddiquee, K, Zhang, S., Guida, WC, Blaskowvich, MA, Greedy, B, Lawrence, HR, Yip, ML, Jove, R., Mclaughlin, MM, Lawrence NJ, Sebti, S., Turkson, J, PNAS, 2007, 104, 7391-6; Siddiquee, KA, Gunning, PT, Glenn, M, Katt, WP, Zhang, S. Schroeck, C, Sebti, SM, Jove, R, Hamilton, AD, Turkson, J, An oxazole-based small-molecule Stat3 inhibitor modulates Stat3 stability and processing and induces antitumor cell effects. ACS Chem Biol. 2007, 2, 787-798.
53. Siddiquee, K.; Zhang, S.; Guida, W.C.; Blaskowvich, M.A.; Greedy, B.; Lawrence, H.R.; Yip, M.L.; Jove, R.; Mclaughlin, M.M.; Lawrence, N.J.' Sebti, S.' Turkson, J. Selective chemical probe inhibitor of Stat3, identified through structure-based virtual screening, induces antitumor activity, PNAS, U S A. 2007, 104, 7391-7396.
54. Stupp, R.; Mason, W.P.; van den Bent, M.J.; Weller, M.; Fisher, B.; Taphoorn, M.J.; Belanger, K.; Brandes, A.A.; Marosi, C.; Bogdahn, U., et al. Radiotherapy plus concomitant and adjuvant temozolomide for glioblastoma. N Engl J Med 2005, 352, 987-996.
55. Sun, S., Yue, P., He, M., Zhang, X., Paladino, D., Al-Abed, Y., Turkson, J., Buolamwini J.K. An Integrated Computational and Experimental Binding Study Identifies the DNA Binding Domain as the Putative Binding Site of Novel Pyrimidinetrione Signal Transducer and Activator of Transcription 3 (STAT3) Inhibitors. Drug Des 2017, 6, 142. doi: 10.4172/2169-0138.1000142.
56. Surawicz, T.S.; Davis, F.; Freels, S.; Laws, E.R., Jr.; Menck, H.R. Brain tumor survival: Results from the national cancer data base. Journal of neuro-oncology 1998, 40, 151-160.
57. Szelag M, Czerwoniec A, Wesoly J, Bluyssen HA. Identification of STAT1 and STAT3 specific inhibitors using comparative virtual screening and docking validation. PLOS One 2015, 10, e0116688.
58. Taillandier, L.; Antunes, L.; Angioi-Duprez, K.S. Models for neuro-oncological preclinical studies: Solid orthotopic and heterotopic grafts of human gliomas into nude mice. Journal of Neuroscience Methods 2003, 125, 147-157.
59. Taylor, T.E.; Furnari, F.B.; Cavenee, W.K. Targeting egfr for treatment of glioblastoma: Molecular basis to overcome resistance. Curr Cancer Drug Targets 2012, 12. 197-209.
60. Turkson J, STAT proteins as novel targets for cancer drug discovery, Expert Opin Ther Targets. 2004, 8, 409-422.
61. Turkson J, Jove R, STAT proteins: novel molecular targets for cancer drug discovery, Oncogene. 2000, 19(56):6613-26.
62. Turkson J, Ryan D, Kim JS, Zhang Y, Chen Z. Haura E, Laudano A, Sebti S, Hamilton AD, Jove R. Phosphotyrosyl peptides block STAT3-mediated DNA binding activity, gene regulation, and cell transformation. J Biol Chem 2001, 276, 45443-45455.
63. Turkson, J., Zhang S, Palmer J, Kay H, Stanko J, Mora LB, Sebti S, Yu H, Jove R. (2004) Inhibition of constitutive.
64. Turkson, J.; Jove, R. Stat proteins: Novel molecular targets for cancer drug discovery. Oncogene 2000, 19, 6613-6626.
65. Verhaak, R.G.; Hoadley, K.A.; Purdom, E.; Wang, V.: Qi, Y.; Wilkerson, M.D.; Miller, C.R.; Ding, L.; Golub, T.; Mesirov, J.P., et al. Integrated genomic analysis identifies clinically relevant subtypes of glioblastoma characterized by abnormalities in pdgfra, idh1, egfr, and nf1. Cancer Cell 2010, 17, 98-110.
66. Yang, C.H.; Murti, A.; Pfeffer, L.M. Stat3 complements defects in an interferon-resistant cell line: Evidence for an essential role for stat3 in interferon signaling and biological activities. Proc Natl Acad Sci U S A 1998, 95, 5568-5572.
67. Yang, C.H.; Murti, A.; Valentine, W.J.; Du, Z.; Pfeffer, L.M. Interferon alpha activates nf-kappab in jak1-deficient cells through a tyk2-dependent pathway. J Biol Chem 2005, 280, 25849-25853.
68. Yang, C.H.; Shi, W.; Basu, L.; Murti, A.; Constantinescu, S.N.; Blatt, L.; Croze, E.; Mullersman, J.E.; Pfeffer, L.M. Direct association of stat3 with the ifnar1 signal transducing chain of the type I IFN receptor. J Biol Chem 1996, 271, 8057-8061.
69 Yang, C.H.; Wang, Y.; Sims, M.; Cai, C.; He, P.; Hacker, H.; Yue, J.; Cheng, J.; Boop, F.A.; Pfeffer, L.M. MicroRna203a suppresses glioma tumorigenesis through an atm-dependent interferon response pathway. Oncotarget 2017, 8, 112980-112991.
70. Yang, C.H.; Wang, Y.; Sims, M.; Cai, C.; He, P.; Yue, J.; Cheng, J.: Boop, F.A.; Pfeffer, S.R.; Pfeffer, L.M. Mirna203 suppresses the expression of protumorigenic STAT1 in glioblastoma to inhibit tumorigenesis. Oncotarget 2016, 7, 84017-84029.
71. Yang, C.H.; Wei, L.; Pfeffer, S.R.; Du, Z.; Murti, A.; Valentine, W.J.; Zheng, Y.; Pfeffer, L.M. Identification of cxcl11 as a stat3-dependent gene induced by ifn. J Immunol 2007, 178, 986-992.
72. Yang, C.H.; Yue, J.; Fan, M.; Pfeffer, L.M. Ifn induces mir-21 through a signal transducer and activator of transcription 3-dependent pathway as a suppressive negative feedback on ifn-induced apoptosis. Cancer Res 2010, 70, 8108-8116.
73. Yang, C.H.; Yue, J.; Pfeffer, S.R.; Handorf, C.R.; Pfeffer, L.M. Microrna mir-21 regulates the metastatic behavior of b16 melanoma cells. J Biol Chem 2011, 286, 39172-39178.
74. Yoo, C.; Kang, J.; Lim, H.Y.; Kim, J.H.; Lee, M.A.; Lee, K.H.; Kim, T.Y.; Ryoo, B.Y. Phase i dose-finding study of opb-111077, a novel stat3 inhibitor, in patients with advanced hepatocellular carcinoma. Cancer Res Treat 2019, 51, 510-518.
75. Yu, H.; Jove, R. The stats of cancer—new molecular targets come of age. Nat Rev Cancer 2004, 4, 97-105.
76. Yue and Turkson, Targeting STAT3 in cancer: how successful are we? Expert Opin Investig Drugs. 2009, 18, 45-56.
77. Yue P, Lopez-Tapia F, Paladino D, Li Y, Chen C-H, Hilliard T, Chen Y, Tius M, Turkson J: Hydroxamic acid and benzoic acid-based STAT3 inhibitors suppress human glioma and breast cancer phenotypes in vitro and in vivo. Cancer Res 2016, 76, 652-663.
78. Yue, P, Lopez-Tapia F, Paladino D, Li Y, Chen CH, Namanja AT, Hilliard T, Chen Y, Tius MA, Turkson J., Hydroxamic Acid and Benzoic Acid-Based STAT3 Inhibitors Suppress Human Glioma and Breast Cancer Phenotypes In Vitro and In Vivo. Cancer Res 2016, 76, 652-663.
79. Zhang X, Yue P, Fletcher S, Zhao W, Gunning PT. Turkson J. A novel small-molecule disrupts STAT3 SH2 domain-phosphotyrosine interactions and STAT3-dependent tumor processes. Biochem Pharmacol 2010, 79, 1398-409.
80. Zhang X, Yue P, Fletcher S, Zhao W, Gunning PT, Turkson J. A novel small-molecule disrupts Stat3 SH2 domain-phosphotyrosine interactions and Stat3-dependent tumor processes. Biochem Pharmacol 2010, 79, 1398-409.
81 Zhang X, Yue P, Page BD, Li T, Zhao W, Namanja AT. Paladino D, Zhao J, Chen Y, Gunning PT, Turkson J. Orally bioavailable small-molecule inhibitor of transcription factor Stat3 regresses human breast and lung cancer xenografts. Proc Natl Acad Sci U S A 2012, 109, 9623-9628.
82. Zhang X, Yue P, Page BDG, et al. Orally bioavailable small-molecule inhibitor of transcription factor Stat3 regresses human breast and lung cancer xenografts. Proc Natl Acad Sci U S A. 2012, 109, 9623-9628.
83. Zhao W, Jaganathan S, Turkson J. A cell-permeable STAT3 SH2 domain mimetic inhibits STAT3 activation and induces antitumor cell effects in vitro. J Biol Chem 2010, 285, 35855-35865.
84. Zhao, G.; Guo, Y.; Chen, Z.; Wang, Y.; Yang, C.; Dudas, A.; Du, Z.; Liu, W.; Zou, Y.; Szabo, E., et al. Mir-203 functions as a tumor suppressor by inhibiting epithelial to mesenchymal transition in ovarian cancer. J Cancer Sci Ther 2015, 7, 34-43.
85. Zhong, Z.; Wen, Z.; Darnell, J.E., Jr. Stat3: A stat family member activated by tyrosine phosphorylation in response to epidermal growth factor and interleukin-6. Science 1994, 264, 95-98.

1. A compound having the structural formula: 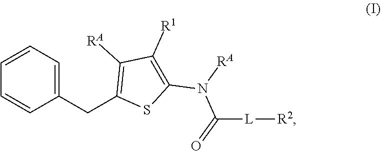
or a pharmaceutically acceptable salt thereof, wherein:
each R^{A} is independently H or methyl;
R¹ is —CN or —C(O)—N(R^{A})₂;
R² is aryl or heteroaryl, wherein the aryl or heteroaryl is monocyclic or bicyclic and comprised of 5-10 ring members, and optionally substituted with 1-4 R^{B}, wherein R^{B} is C₁-C₂ alkyl, fluoro, chloro, or —O—(C₁-C₂ alkyl), wherein each alkyl is optionally fluorinated.
L is absent or —C(R^{A})₂—O—.

2. The compound of claim 1, wherein R¹ is —CN or —C(O)—NH₂.

3. The compound of claim 1, wherein R¹ is —CN.

4. The compound of claim 1, wherein R² is phenyl or benzofuranyl.

5. The compound of claim 4, wherein R² is phenyl.

6. The compound of claim 1, wherein R² is unsubstituted.

7. The compound of claim 1, wherein R² is substituted with 1-2 R^{B}, wherein R^{B} is CH₃, CF₃, chloro, —O—CH₃, or —O—CF₃.

8. The compound of claim 1, wherein L is absent.

9. The compound of claim 1, wherein L is —C(R^{A})₂—O—.

10. The compound of claim 9, wherein L is —C(CH₃)₂—O— or —CH₂—O—.

11. The compound of claim 9, wherein L is —CH(CH₃)—O—.

12. The compound of claim 1, wherein the compound is not 5-benzyl-2-(2-(2-methoxyphenoxy)acetamido)-4-methylthiophene-3-carboxamide, N-[3-carbamoyl-5-benzyl-thien-2-yl]4-hydroxyphenoxyacetamide, or 5-benzyl-4-methyl-2-(2-phenoxyacetamido)thiophene-3-carboxamide.

13. The compound of claim 1, wherein the compound is selected from a group consisting of N-(5-benzyl-3-cyanothiophen-2-yl)-2-phenoxyacetamide, N-(5-benzyl-3-cyanothiophen-2-yl)-2-phenoxypropanamide, N-(5-benzyl-3-cyanothiophen-2-yl)-2-(m-tolyloxy)acetamide, 5-benzyl-2-(2-(2,6-dimethylphenoxy)acetamido)-4-methylthiophene-3-carboxamide, N-(5-benzyl-3-cyanothiophen-2-yl) benzofuran-2-carboxamide, N-(5-benzyl-3-cyanothiophen-2-yl)-2-(3-(trifluoromethyl)phenoxy)acetamide, N-(5-benzyl-3-cyanothiophen-2-yl)-2-methyl-2-phenoxypropanamide, N-(5-benzyl-3-cyanothiophen-2-yl)-2-(4-(trifluoromethoxy)phenoxy)acetamide, N-(5-benzyl-3-cyanothiophen-2-yl)-2-(4-chlorophenoxy)acetamide, and 5-benzyl-4-methyl-2-(2-phenoxypropanamido)thiophene-3-carboxamide.

14. A method of treating cancer in a subject in need thereof, comprising administering a therapeutically effect amount of a compound according to claim 1.

15. The method of claim 14, wherein the cancer is selected from breast cancer, glioblastoma multiforme brain cancer, colorectal cancer, lung cancer, pancreatic cancer, bladder cancer, and metastatic prostate cancer.
